(19) United States
(12) Patent Application Publication
(10) Pub. No.: US 2013/0178940 A1
(43) Pub. Date: Jul. 11, 2013

(54) EXPANDABLE CAGE SPINAL IMPLANT

(71) Applicant: Thompson MIS, Salem, NH (US)

(72) Inventor: Daniel K. Farley, Traverse City, MI (US)

(73) Assignee: Thompson MIS, Salem, NH (US)

(21) Appl. No.: 13/735,086

(22) Filed: Jan. 7, 2013

Related U.S. Application Data
(60) Provisional application No. 61/583,894, filed on Jan. 6, 2012.

(51) Int. Cl. A61F 2/44

(52) U.S. Cl. CPC A61F 2/442
USPC 623/17.16

(57) ABSTRACT

An expandable cage for maintain the position of adjacent vertebrae includes a cage top, a cage bottom, and a plurality of shims. The cage top is adapted for contacting a bottom surface of the upper of the adjacent vertebrae, and the cage bottom is adapted for contacting an upper surface of the lower of the adjacent vertebrae. The plurality of shims are adapted for insertion between the cage top and cage bottom to expand the cage. The shims include a proximal end having a bone graft distribution feature configured such that when bone graft material is introduced to the site of the expandable cage, the bone graft material is separated at the bone graft distribution feature and distributed to either side of the cage.

### CROSS-REFERENCE TO RELATED APPLICATION

None.

### STATEMENT REGARDING FEDERALLY SPONSORED RESEARCH OR DEVELOPMENT

None.

### BACKGROUND OF THE INVENTION

The present invention relates to systems and methods for providing spinal implants, for example, to be used in connection with spinal fusion.

Spinal fusion is a surgical procedure that fuses two or more vertebrae together using bone graft materials supplemented with devices. Spinal fusion may be performed for the treatment of chronic neck and/or back pain, trauma, and neoplasms. Spinal fusion can be used to stabilize and eliminate motion of vertebrae segments that may be unstable, or move in an abnormal way, that can lead to discomfort and pain. Spinal fusion may be performed to treat injuries to the vertebrae, degeneration of spinal discs, abnormal spinal curvature, and/or a weak or unstable spine.

Spinal fusion generally requires a graft material, usually bone material, to fuse the vertebrae together. The bone graft material can be placed over the spine to fuse adjacent vertebrae together. Alternatively, a device (i.e. cage) may be positioned between the vertebrae being fused and filled with the bone graft material. Such a cage can include holes that allow the vertebrae and the graft material to grow together to provide fusion, with the cage supporting the weight of the vertebrae while the fusion is occurring. Most of these cages are limited to only a few cubic centimeters of bone graft material thus limiting the fusion area achieved. Because the fusion mass is under pressure, fusion can be promoted. The disc space height can be restored, taking pressure off of the nerves. The spine alignment, foraminal height, and canal diameter can be restored. In some cases the graft can be placed with minimal disruption of muscles and ligaments using minimally invasive approaches to the spine, thus preserving the normal anatomical integrity of the spine. Other interbody device assemblies are also presently known.

For example, certain interbody devices are inserted directly between two vertebrae. For designs that are inserted at their full height, these may be difficult to insert and/or distract vertebrae more than desired due to the curvature of the vertebral surfaces. Certain known interbody devices are inserted sideways (at a lower height) and then rotated (to a greater height) to ease insertion while still allowing for the desired distraction. These include those disclosed in U.S. patent application Ser. Nos. 11/623,356, filed Jan. 16, 2007, titled “Minimally Invasive Interbody Device,” and 11/932,175, filed Oct. 31, 2007, titled “Minimally Invasive Interbody Device Assembly,” which are hereby incorporated by reference in their entirety.

While these designs allow for eased insertion, these designs typically have fixed dimensions, thereby requiring a certain amount of trial and error and/or not providing for any adjustability, beyond removing a spacer and replacing it with a differently sized spacer. Certain known designs provide some flexibility by providing a series of elements aligned as a column, with the elements added serially until a desired height is reached. An example of such a device is disclosed in U.S. Published Application No. 2011/0009870, titled “Apparatus for Spinal Fusion.” These designs, however, have limitations regarding distribution of bone graft material, as they may fill the access hole created for their deployment and not allow room for insertion of bone graft material. Further, these designs may also require shaving vertebral bodies down to a generally flat surface, which can remove more of the vertebral body than desirable.

Typically, the bone graft material is autogenous bone material taken from the patient, or allograft bone material harvested from a cadaver. Synthetic bone material can also be used as the graft material. Generally, the patient's own bone material offers the best fusion material since it offers osteoinductive, osteoconductive, and osteogenesis properties. Known bone fusion. materials include iliac crest harvest from the patient, bone graft extenders, such as hydroxyapetite and demineralized bone matrix, and bone morphogenic protein.

Minimally invasive surgical procedures have been devised in an attempt to preserve normal anatomical structures during spinal surgery. Many known procedures for spinal fusion, however, still are more invasive than desired. Additionally, many known procedures do not provide the level of control over the delivery and placement of the bone graft material as could be desired. Additionally, current interbody devices only allow for a limited application of bone material (i.e., cages), and because of their relative size place the neural elements at risk during placement, often resulting in undersized implants being placed.

It is therefore one object of the present invention to provide a spinal implant system that reduces approach related morbidity, allows for more bone graft placement and/or provides improved control of the delivery and/or placement of bone graft material.

### BRIEF SUMMARY OF THE INVENTION

These and other objects of the invention are achieved in an expandable cage for maintaining the position of adjacent vertebrae. The expandable cage includes a cage top, a cage bottom, and a plurality of shims. The cage top is adapted for contacting a bottom surface of the upper of the adjacent vertebrae, and the cage bottom is adapted for contacting an upper surface of the lower of the adjacent vertebrae. The plurality of shims are adapted for insertion between the cage top and cage bottom to expand the cage. The shims include a proximal end having a bone graft distribution feature configured such that when bone graft material is introduced to the site of the expandable cage, the bone graft material is separated at the bone graft distribution feature and distributed to either side of the cage. For example, the proximal end of the shims may have a leading edge including tapered surfaces that lead to a generally sharp point.

Certain embodiments of the present invention provide an expandable cage system including a cage and a funnel. The cage includes a cage top, a cage bottom, and a plurality of shims. The cage top is adapted for contacting a bottom surface of the upper of the adjacent vertebrae, and the cage bottom is adapted for contacting an upper surface of the lower of the adjacent vertebrae. The plurality of shims are adapted for insertion between the cage top and cage bottom to expand the cage. The shims include a proximal end having a leading edge including tapered surfaces that lead to a generally sharp point. The funnel defines a conduit through which bone graft material may passed from an external source to the implanted cage system. The funnel includes a delivery end that is positioned proximate to the proximal end of the plurality of shims during the delivery of bone graft material. The delivery end of the funnel may include a notch sized and configured to be accepted by the leading edge of the plurality of shims.

### BRIEF DESCRIPTION OF THE SEVERAL VIEWS OF THE DRAWINGS

FIG. 1 illustrates a side view of an expandable cage system formed in accordance with an embodiment of the present invention, without shims in place.

FIG. 2 illustrates an end view of the system of FIG. 1.

FIG. 3 illustrates a top view of the system of FIG. 1.

FIG. 4 illustrates a side view of an expandable cage system, with shims in place, and without sides.

FIG. 5 illustrates a top view of a shim formed in accordance with an embodiment of the present invention.

FIG. 6 illustrates a side view of the shim of FIG. 5.

FIG. 7 illustrates a top view of an inserter formed in accordance with an embodiment of the present invention.

FIG. 8 illustrates a top partial view of a funnel formed in accordance with an embodiment of the present invention.

FIG. 9 illustrates an overhead view taken through a section of a patient's vertebral column showing distribution of bone graft material around a cage system formed in accordance with an embodiment of the present invention.

### DETAILED DESCRIPTION

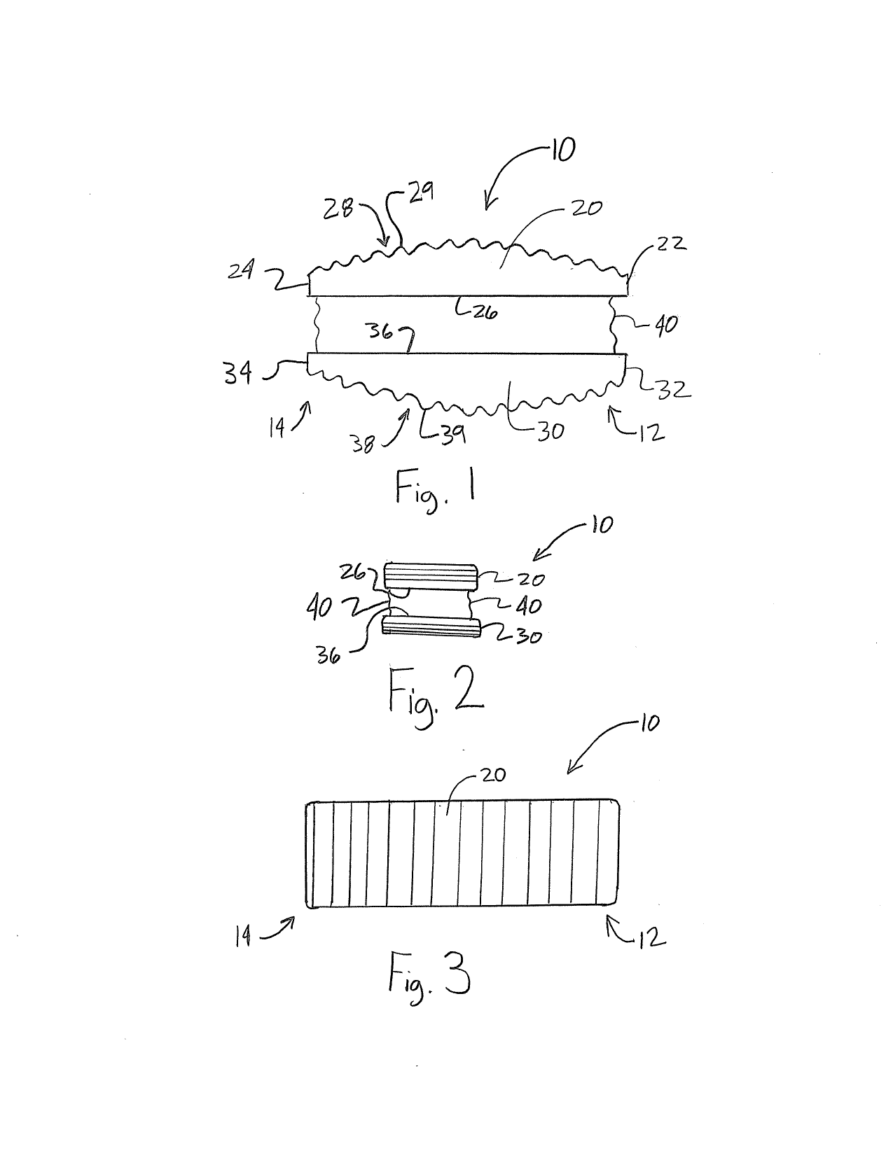

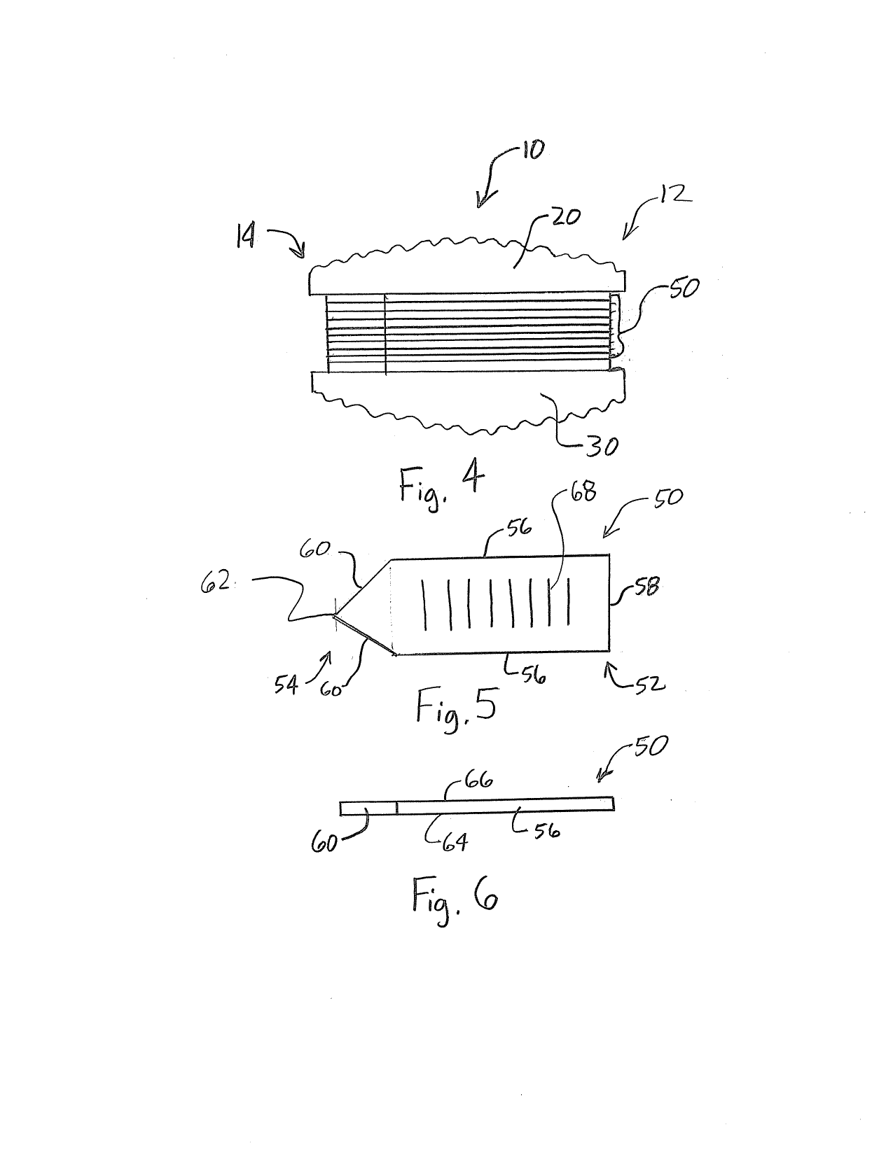

FIG. 1 illustrates a side view of an expandable cage system 10, without shims in place, formed in accordance with an embodiment of the present invention, FIG. 2 illustrates an end view of the system 10, FIG. 3 illustrates a top view of the system 10, and FIG. 4 illustrates a side view of the system 10, with shims in place (the system as illustrated in FIG. 4 does not include sides). The expandable cage system 10 includes a cage top 20, a cage bottom 30, sides 40, and shims 50. The expandable cage system 10 is adapted to be inserted between two vertebrae to be distracted, with the cage top 20 oriented toward the bottom surface of a first, higher vertebrae and the cage bottom 30 oriented toward the top surface of a second, lower vertebrae. Shims 50 are then added to urge the cage top 20 and cage bottom 30 into contact with the vertebrae and to distract the vertebrae to a desired position. Once the desired spacing has been achieved, bone graft material may be added around the system 10. Viewed from above (e.g., FIG. 3), the cage system of the illustrated embodiment defines a generally rectangular shape. Other embodiments may define other shapes, such as rounded sides and/or ends, and/or features such as radii, chambers, or bevels proximate to the corners. The various cage components may be made from a variety of materials. For example, PEEK (polyether ether ketone), titanium, carbon fiber, bone allograft, or a plurality of materials may be used in components of the cage system.

As seen in FIG. 1, the expandable cage system 10 includes a cage top 20 and a cage bottom 30 joined by sides 40. In the illustrated embodiment, the cage top 20 is shaped to generally configured to match with the lower surface of the upper vertebrae of the two vertebrae, between which the expandable cage system 10 is inserted. Similarly, the cage bottom 30 is generally configured to match with the upper surface of the lower vertebrae of the two vertebrae between which the expandable cage system 10 is inserted. The expandable cage system 10 includes a distal end 12 configured to be oriented more deeply inside the patient when inserted during a procedure, and a proximal end 14 configured to be oriented toward the practitioner when the system 10 is inserted into a patient during a procedure. The sides 40 may extend only along a portion of the length of the cage top 20 and cage bottom 30, and/or may be slotted or perforated to allow bone graft material to pass through the sides. The sides 40 may be sufficiently elastic and/or include bellows to allow the cage top 20 and cage bottom 30 to be biased apart from each other while still being connected by the sides. In other embodiments, the sides may be one or more ribbons or strings that connect the cage top 20 and bottom 30 spaced at distances apart to allow the flow of bone graft material through the sides. In still other embodiments, sides may not be provided, with the cage top and bottom unconstrained laterally with respect to each other than by frictional forces between the cage top and bottom and the vertebral bodies and/or frictional forces between the cage top and bottom and the shims. In yet further embodiments, the cage top, bottom, and shims may include features such as rails; slots, or other guides to help maintain lateral alignment of the top, bottom, and shims.

The cage top 20 includes a distal end 22 and a proximal end 24, as well as a bottom surface 26 configured to contact the shims 50 when the expandable cage system 10 is in an expanded position caused by the insertion of the shims 50. The cage top 20 also includes a top surface 28 that includes features 29. As indicated above, the top surface 28 is formed to generally match with a shape of the vertebral surface that it contacts. In the illustrated embodiment, the top surface 28 is radiused to have a greater height at a central portion than at either the distal end 22 or the proximal end 24, and is generally symmetric from end to end and from side to side. The top surface 28 may, for example, be about 28 millimeters long by about 9 millimeters wide. In alternate embodiments, different sizes and/or shapes may be used to suit different patients and/or procedures. For example, the distal end 22 may have a greater height than the proximal end 24 for certain procedures to correct a lordosis. As another example, the top surface may be radiused from side to side to match certain vertebral contours. Such radiusing may be symmetric or asymmetric, and the radius may vary at different points along the length of the top surface as well. In the illustrated embodiment, the top surface 28 includes features 29 that may include for example, ridges and/or pyramids and/or teeth and/or notches and/or grooves, that provides for improved grip along the vertebral surface to help secure the expandable cage system 10 in place while shims 50 are being added, and/or when bone graft material is being added to the site of interest, and/or after the procedure has been completed. Further, in certain embodiments, the features may allow an amount of bone graft material to fill in spaces between the top surface and the vertebral surface.

The cage bottom 30 includes a distal end 32 and a proximal end 34, as well as an upper surface 36 configured to contact the shims 50 when the expandable cage system 10 is in an expanded position caused by the insertion of the shims 50. The cage bottom 30 also includes a bottom surface 38 that includes features 39. As indicated above, the bottom surface 38 is formed to generally match with a shape of the vertebral surface that it contacts. In the illustrated embodiment, the bottom surface 38 is radiused to have a greater height at a central portion than at either the distal end 32 or the proximal end 34, and is generally symmetric from end to end and from side to side. The bottom surface 38 may, for example, be about 28 millimeters long by about 9 millimeters wide. In alternate embodiments, different sizes and/or shapes may be used to suit different patients and/or procedures. For example, the distal end 32 may have a greater height than the proximal end 34 for certain procedures to correct a lordosis. As another example, the bottom surface may be radiused from side to side to match certain vertebral contours. Such radiusing may be symmetric or asymmetric, and the radius may vary at different points along the length of the top surface as well. In the illustrated embodiment, the bottom surface 38 has a generally similar contour as the top surface 28, and is generally symmetric with the top surface 28. In certain embodiments, however, the top surface 28 and bottom surface 38 may not be symmetric, and may have differently configured contours, sizes, and/or proportions.

Similar to the top surface 28, in the illustrated embodiment, the bottom surface 38 includes features 39 that may include for example, ridges and/or pyramids and/or teeth and/or notches and/or grooves, that provides for improved grip along the vertebral surface to help secure the expandable cage system 10 in place while shims 50 are being added, and/or when bone graft material is being added to the site of interest, and/or after the procedure has been completed. Further, in certain embodiments, the features may allow an amount of bone graft material to fill in spaces between the top surface and the vertebral surface.

FIG. 5 illustrates a top view of a shim 50 formed in accordance with an embodiment of the present invention, and FIG. 6 illustrates a side view of the shim 50. The shim 50 includes a distal end 52 (corresponding to the distal end 12 of the cage system 10) and a proximal end 54 (corresponding to the proximal end 14 of the cage system 10). The shim 50 includes sides 56 spaced, proximate to the distal end 52, by a front edge 58. In the illustrated embodiment, the sides 56 are spaced apart such that the shim 50 has a width approximately the same as the cage top 20 and cage bottom 30. In alternate embodiments, the shims may be wider or narrower. In the illustrated embodiment, the front edge 58 extends generally. perpendicularly to the sides 56 along the length of the front edge 58. The front edge 58 of the shim 50, along with corresponding portions of the cage top 20 and cage bottom 30, may, in alternate embodiments, define a different shape, such as being more rounded and/or having chamfers or bevels proximate to their ends where they meet the sides 56 of the shims 50.

Proximate to the proximal end 54 of the shim 50, tapered surfaces 60 extend generally proximally as well as inwardly from the sides 56. The tapered surfaces 60 extend substantially inwardly to provide a geometry configured to separate introduced bone graft material and direct the introduced bone graft material to either side of the shim 50 and cage system 10. In the illustrated embodiment, the tapered surfaces 60 extend toward and meet at a point located substantially along a center line of the shim 50, thereby forming a sharp point 62. The tapered surfaces 60 and sharp point 62 are an example of a wedge-shaped leading edge, and also provide an example of a bone graft distribution feature. Other wedge-shaped leading edges may be employed in alternate embodiments, such as a more rounded tip than shown in the illustrated embodiment. In alternate embodiments, the tapered surfaces 60 may not meet at a sharp point but may include a small flat area, however, the flat portion should be kept appropriately small to insure the configuration still effectively separates the bone graft material to either side of the cage system.

The shim 50 also includes a bottom 64 and a top 66. The distance between the bottom 64 and top 66 defines the height of the shim 50. The height may be, for example, about one millimeter. Thus, a series of wedges may be inserted into the cage system one at a time, allowing adjustment in increments of about one millimeter. In alternate embodiments, thinner or thicker shims, or a combination of different thicknesses, may be used to provide different adjustment increments. Further, in certain embodiments, the thickness of the shim may be tapered, sloped, or wedged to provide a leading sloped surface across the height or a portion of the height of the shim at the distal and/or proximal end of the shim to ease the insertion of shims into the expandable cage.

As seen in FIG. 5, the top 66 includes ridges 68. In the illustrated embodiment, the ridges 68 are spaced apart and extend generally horizontally across a portion of the width of the shim 50. In other embodiments, the ridges may be arranged differently, such as in a staggered pattern, and/or as a series of shorter ridges with space between adjacent ridges both lengthwise and widthwise across the shim, and/or in a grid or matrix type pattern, and/or oriented differently, such as extending along the length of the shim instead of across the width, or, as another example, extending diagonally. The ridges 68 are an example of a stabilizing feature. The ridges 68 act to help secure the shim 50 in place relative to adjacent shims and/or the bottom surface 26 of the cage top 20 and/or the top surface 36 of the cage bottom 30. The ridges 68 may be made of the same material as the shim 50, or may be a different material. In some embodiments, stabilizing features are located only on the top of the shim, in others only on the bottom, and in other embodiments stabilizing features are located on both the top and bottom of the shim. Further still, in certain embodiments, the stabilizing features are different on the top and bottom, and may be complementary or cooperating. For example, the top of the shims may include tabs while the bottom of the shims may include slots configured to accept the tabs. Further, cooperating features may also be provided on the bottom surface of the cage top and/or the top surface of the cage bottom for improving stability when in contact with the shims. The stabilizing features are preferably configured to help maintain the shims in position while not inappropriately impeding or preventing the sliding into and positioning of the shims in the cage system between the cage top and cage bottom.

The expandable cage system 10 may also include an inserter configured to facilitate insertion of the shims 50 between the cage top 20 and the cage bottom 30. An inserter formed in accordance with an embodiment of the present invention is illustrated in FIG. 7.

The inserter 70 includes a handle end 72 and an inserter end 74. The handle end 72 is oriented proximally and includes a handle 76 configured for grasping by a practitioner. The inserter end 74 is oriented distally and includes and insertion feature 76 configured to interact with one or more shims 50 for insertion into the cage system 10 and/or for manipulation of a shim 50 and/or the cage system 10 after a shim 50 is partially or fully inserted. In the illustrated embodiment, the insertion feature 76 includes sloped surfaces 78 corresponding to the tapered surfaces 60 of the shim 50 and forming an opening configured to accept at least a portion of the proximal portion of the shim 50.

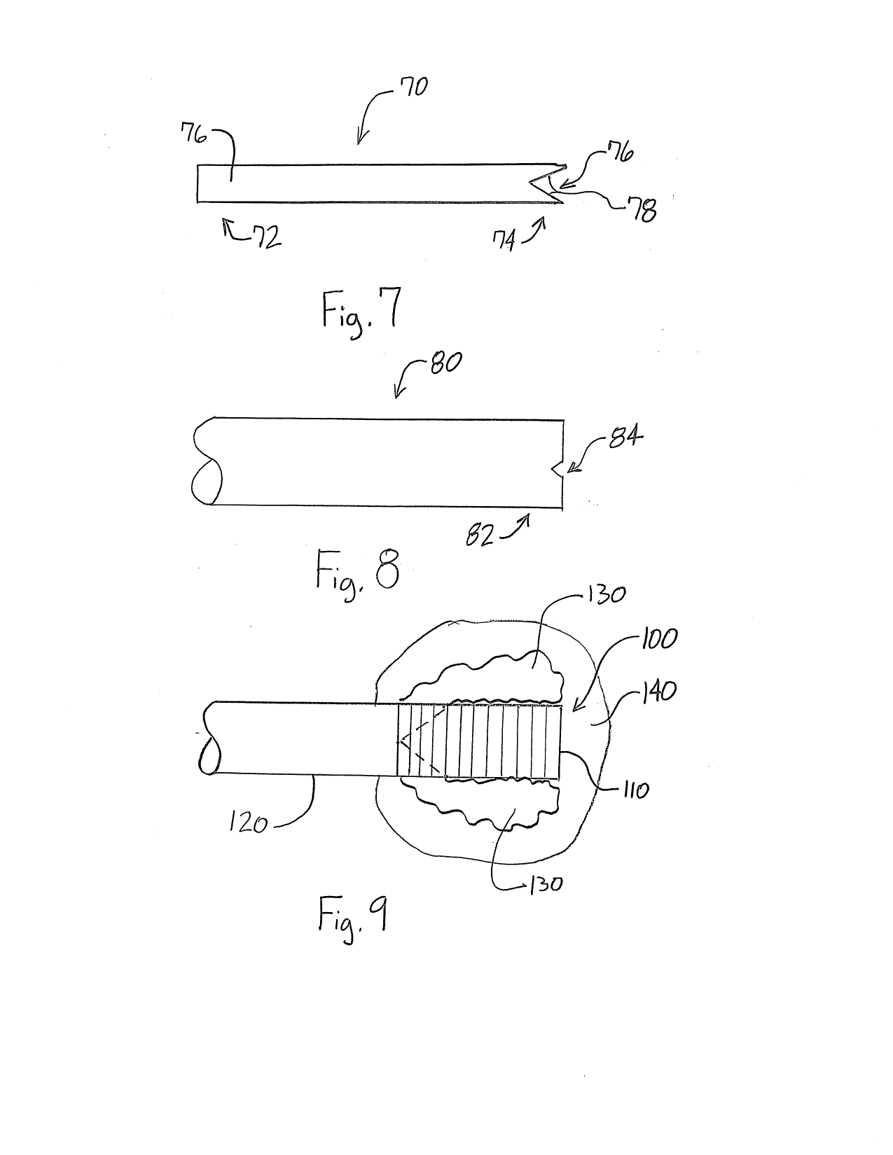

The expandable cage system 10 may also include a funnel 80 configured to help provide bone graft material toward the cage system 10 and surrounding anatomy. FIG. 8 illustrates a top partial view of a funnel 80 formed in accordance with an embodiment of the present invention. The funnel 80 defines a conduit through which bone graft material may be passed, and includes a delivery end 82 and a notch 84. The delivery end 82 is positioned at or near to the proximal end 14 of the cage system 10 after the cage system 10 is positioned and configured as desired in place in a patient. Bone graft material is passed through the funnel 80 and out of the delivery end 82 to the site of interest surrounding the implanted cage system 10. The notch 84 is sized and configured to be accepted by a feature at the proximal end of one or more shims 50 (for example by the sharp point 62 and potions of the tapered surfaces 62) positioned in the cage system 10. In the illustrated embodiment, the overall height of the funnel is selected to correspond generally to the overall height of the shims, so that the notch contacts the shims instead of the top or bottom of the cage. In alternate embodiments, the funnel may have a larger overall height, and/or the cage top or bottom may include an alignment feature cooperating with the funnel. The notch 84 helps to position and/or stabilize the funnel 80 with respect to the cage system 10. In alternate embodiments, the funnel may not have a notch. Funnels of different embodiments may be various shapes in cross-section. Funnels, for example, may have generally circular cross-sections, generally square cross-section, generally rectangular cross-sections, or generally oval, egg-shaped or otherwise oblong round cross-sections. Further still, funnels may be symmetric or asymmetric about the center of the funnel and/or the center of the cage system. For example, a funnel may be configured to distribute bone graft material generally equally to each side of the cage system, to distribute bone graft material exclusively to one side of the cage system (or one side at a time), or to distribute a first amount or rate of bone graft material to one side of the cage system and a second larger amount or rate to the other side.

An example of the use of an expandable cage system will now be discussed with reference to FIG. 9, which illustrates an overhead view taken through a section of a patient's vertebral column showing distribution of bone graft material 130 around a cage system 100 formed in accordance with an embodiment of the present invention.

The cage system 100 of the illustrated embodiment includes an expandable cage 110 and a funnel 120, and may be generally similar to the above discussed embodiments. The expandable cage 110 is designed to be implanted in a disc space 140 to establish a desired spacing between adjacent vertebrae.

To implant the expandable cage 110, first an incision is made for access to the site of interest, and the site of interest, including the vertebral bodies, prepared. Because the expandable cage 110 of the illustrated embodiment is contoured to generally match the surface contour of the vertebral bodies, less time preparing the bodies, such as scraping material from the vertebral bodies, is required, and less material is removed. With the site of interest prepared, the top and bottom of the expandable cage 110 are inserted into the site, with the top oriented toward the bottom surface of the higher of the vertebrae, and the bottom oriented toward the upper surface of the lower of the vertebrae, and positioned as desired. At insertion, the top and bottom may be inserted without any shims between them. Alternatively, if the gap between the vertebral bodies at insertion is sufficiently large so that the top and bottom, along with a given number of shims, may be inserted between the vertebrae while still maintaining a clearance between the expandable cage 100 and the vertebral bodies, then a number of shims may be inserted with the top and bottom of the cage, with the shims already between the top and bottom.

With the expandable cage 110 inserted and positioned as desired, additional shims are then added to expand the expandable cage 110 and thereby also position the vertebral bodies at the desired spacing. The shims are added with their bone graft distribution feature positioned proximally. With the vertebral bodies spaced and positioned as desired, bone graft material may now be introduced to fill in the open volume around the cage, for example, on either side of the cage, and to provide fusion, and improve the stability of the implant site. To provide the bone graft material to the site of interest, the funnel 120 is positioned with a delivery end proximate to the proximal end of the cage and the bone graft distribution feature of the shims, and bone graft material 130 passed through the funnel 120 to the site of interest. For example, a tamping rod (not shown) may be used to advance the material through the funnel. As the bone graft material 130 meets the bone graft distribution feature (for example, a wedge with a generally sharp leading edge), the bone graft material is separated and distributed to each side of the cage as it passes along and past the bone graft distribution feature. After a desired amount of bone graft material has been added and positioned in the site of interest, the funnel may be withdrawn.

In certain embodiments, an expandable cage system may be adapted for and used for vertebral body replacement. In such embodiments, the cage would be placed within the general outline of a single vertebral body that has been damaged, for example crushed, and used to help return the vertebral body to its original height. For example, when a vertebral body is crushed, the cage can be inserted into the center of the body and expanded to the body's original height, with bone and/or bone substitutes and/or bone cement injected to help rebuild the vertebrae. The cage top for such a cage system would not need to be configured to correspond to a lower surface of the higher of two vertebrae as discussed above, but instead the cage top could engage an upper portion of the vertebral body being repaired. Similarly, the cage bottom could engage a lower portion of the vertebral body being repaired.

In certain embodiments of the present invention, a kit is provided including a variety of sizes and/or types of cage top and bottoms, and/or a variety of sizes of shims to accommodate different patients and procedures.

While particular embodiments of the invention have been shown, it will be understood that the invention is not limited thereto since modifications may be made by those skilled in the art, particularly in light of the foregoing teaching. It is therefore, the appended claims that define the true spirit and scope of the invention.

What is claimed is:

1. An expandable cage for maintaining the position of adjacent vertebrae, the cage including:
a cage top adapted for contacting a bottom surface of the upper of the adjacent vertebrae;
a cage bottom adapted for contacting an upper surface of the lower of the adjacent vertebrae; and
a plurality of shims adapted for insertion between the cage top and cage bottom for expanding the cage, wherein the shims include a proximal end having a bone graft distribution feature configured to separate and distribute introduced bone graft material to either side of the cage.

2. An expandable cage for maintaining the position of adjacent vertebrae, the cage including:
a cage top adapted for contacting a bottom surface of the upper of the adjacent vertebrae;
a cage bottom adapted for contacting an upper surface of the lower of the adjacent vertebrae; and
a plurality of shims adapted for insertion between the cage top and cage bottom for expanding the cage, wherein the shims include a proximal end having a leading edge including tapered surfaces leading to a generally sharp point, the leading edge configured to separate and distribute introduced bone graft material to either side of the cage.

3. An expandable cage system for maintaining the position of adjacent vertebrae, the cage system including:
a cage including
a cage top adapted for contacting a bottom surface of the upper of the adjacent vertebrae;
a cage bottom adapted for contacting an upper surface of the lower of the adjacent vertebrae; and
a plurality of shims adapted for insertion between the cage top and cage bottom for expanding the cage, wherein the shims include a proximal end having a leading edge including tapered surfaces leading to a generally sharp point, the leading edge configured to separate and distribute introduced bone graft material to either side of the cage; and
a funnel defining a conduit through which bone graft material may be passed, the funnel including a delivery end configured to be positioned proximate to the proximal end of the plurality of shims during bone graft delivery, the delivery end including a notch sized and configured to be accepted by the leading edge of the plurality of shims.

4. An expandable cage system for vertebral body replacement, the cage system including:
a cage top for engaging an upper portion of a vertebrae body;
a cage bottom for engaging a lower portion of the vertebrae body; and
a plurality of shims adapted for insertion between the cage top and bottom for expanding the cage, wherein the shim include a proximal end having a bone graft distribution feature configured to separate and distribute introduce bone graft material to either side of the cage.
